(19) United States
(12) Patent Application Publication
(10) Pub. No.: US 2024/0149081 A1
(43) Pub. Date: May 9, 2024

(54) Devices and methods to relieve dry eye symptoms

(71) Applicants: Tianxin Wang, Walnut Creek, CA (US); Leonardo Wang, Walnut Creek, CA (US)

(72) Inventors: Tianxin Wang, Walnut Creek, CA (US); Leonardo Wang, Walnut Creek, CA (US)

(73) Assignee: Tianxin Wang, Walnut Creek, CA (US)

(21) Appl. No.: 18/386,598

(22) Filed: Nov. 2, 2023

Related U.S. Application Data
(60) Provisional application No. 63/423,027, filed on Nov. 6, 2022.

(51) Int. Cl. A62B 18/02; A61F 9/00

(52) U.S. Cl. CPC A62B 18/025; A61F 9/00

(57) ABSTRACT

Methods and devices with noninvasive means for providing moisture from breath to the eyes and nose, for relieving dry eye symptoms and relieving dry nose symptoms, and to hydrate the eyes, nose and facial skin are described. The device for relieving dry eye symptoms comprises a face covering over the nose, a breath deflector and a means that can attach the device to the face.

### CROSS-REFERENCE TO RELATED APPLICATIONS

This application claims benefit of and priority to U.S. Provisional Patent Application Ser. No. 63/423,027 filed on Nov. 6, 2022, which is incorporated herein by reference in its entirety for all purposes. The entire disclosure of the prior application is considered to be part of the disclosure of the instant application and are hereby incorporated by reference.

### SUMMARY OF THE INVENTIONS

This application discloses methods and devices with non-invasive means for providing moisture from breath to the eyes, nose and face, for relieving dry eye symptoms and relieving dry nose symptoms, and to hydrate the eyes, nose and facial skin. The device for relieving dry eye symptoms comprises a face covering over the nose, a breath deflector and a means that can attach the device to the face.

### BACKGROUND

Dry eye is the condition of having dry eyes. Other associated symptoms include irritation, redness, discharge, and easily fatigued eyes. Blurred vision may also occur. Symptoms range from mild and occasional to severe and continuous. Dry eye occurs when either the eye does not produce enough tears or when the tears evaporate too quickly. This can result from contact lens use, meibomian gland dysfunction, pregnancy, Sjögren syndrome, vitamin A deficiency, omega-3 fatty acid deficiency, LASIK surgery, and certain medications such as antihistamines, some blood pressure medication, hormone replacement therapy, and antidepressants. Diagnosis is mostly based on the symptoms, though a number of other tests may be used. Treatment depends on the underlying cause. Artificial tears are usually the first line of treatment. Dry eye syndrome is a common eye disease. It affects 5-34% of people to some degree depending on the population looked at. Among older people it affects up to 70%.

Dry nasal passages are typically caused by a lack of mucus production in the sinuses, which can leave the nose feeling dry and irritated. Mucous membranes, which line the sinuses and keep them moist, make mucus that is supposed to easily flow through the nasal passageways. However, dry conditions can prevent mucus from flowing freely and may lead to problems. Dry sinuses occur when the mucous membranes in sinuses lack proper moisture. This can lead to dry nasal passages, discomfort, nosebleeds, and similar unpleasant symptoms. In severe cases, untreated dry sinuses can become infected and require antibiotics. Dry sinuses can cause many uncomfortable symptoms including sore throat, headache, sinus pain or pressure, nosebleeds, dry nose and dry mouth.

Skin hydration prevents wrinkles & aging skin. Improving skin's hydration levels keep skin healthy and rejuvenated. The outermost layer of the skin has the moisture it needs to flush away toxins and carry nutrients to the skin's cells. Skin that is well-hydrated also is less sensitive to irritants and germs. Keeping skin hydrated can protect the face from premature appearance of wrinkles and acne.

### BRIEF DESCRIPTION OF THE DRAWINGS

FIG. 1 shows examples of a device having a convex face covering structure and temples to be mounted on the ears.

FIG. 2 shows an example of a device is worn by the user.

FIG. 3 shows examples of the device made of transparent rigid material.

FIG. 4 shows additional examples of the device with different shapes having either temples or head bands/strings/straps as mounting means.

FIG. 5 shows additional examples of the device with different shapes having either temples or head bands/strings/straps worn by the user.

FIG. 6 shows additional examples of the device having the area covering eyes open.

FIG. 7 shows examples of the device having both airflow blocking means and breath deflector.

FIG. 8 shows different views of FIG. 7c.

FIG. 9 shows longitudinal plane views of examples of the user wearing the device.

FIG. 10 shows the face covering of the device can further extend down to or cover the mouth.

FIG. 11 shows examples of one-way valve/opening can be placed to the area close to the nose.

FIG. 12 shows additional examples of the device being used by the user.

FIG. 13 shows examples of the device having a glasses rack and deflector.

FIG. 14 shows example of face covering can include a nose protrusion area or conic structure in front of the nose.

FIG. 15 shows example of device using a flexible sheet as face cover.

FIG. 16 shows example of a device to relieve dry nose symptoms and hydrate respiratory duct.

FIG. 17 shows examples of device to hydrate facial skin.

FIG. 18 shows examples of device having see through area in front of the eyes equipped with groove/pocket frame that allow glass/lenses to be inserted.

FIG. 19 shows the method to make custom fit device for a user.

### DETAILED DESCRIPTION

The current invention discloses devices for providing moisture to eyes and relieving dry eye symptoms. The device comprises a face covering over the nose, a breath deflector and a means that can attach the device to the face. The breath deflector deflects the exhaled breath from the nose to the upper portion of the face covering, thus causing the moisture in the breath to reach the eyes to moisturize the eyes/relieve dry eye symptoms. The face covering over the nose and eye area or its surrounding areas provides an air channel/passage/duct that allows a significant amount of moisture to be able to reach the eyes. The face covering can use one type of material or use different types of material at different areas. The deflector or the edge of the deflector can be wrapped with or be made of soft material such as rubber, foam or silicone.

The current invention discloses methods for providing moisture to the eyes and relieving dry eye symptoms. The method involves delivering the user's exhaled breath to his or her eye area to provide moisture there to relieve dry eyes. The exhaled breath contains significant amount of moisture. The method comprises attaching the device described in the current invention to the face to deliver moisture from the breath to the eyes to moisturize the eyes/relieve dry eye symptoms. A user mounts the device of the current invention on his face as shown in the figures and breathes, therefore delivers the moisture containing exhaled breath to his eyes to relive dry eyes.

In some embodiments, the face covering covers the nose, eyes and the facial area between them, and does not cover the mouth. In some embodiments, the face covering covers the nose, the area close to the lower eyelid and the facial area between them, and does not cover the mouth and eyes. In some embodiments, the face covering is made of transparent material such as plastic and glass. In some embodiments, the face covering is made of rigid material. In some embodiments, the face covering uses different materials in different areas. For example, the area covering eyes can use a transparent material such as anti-scratch glass with anti-fog coating while other parts can use rigid or semi-rigid or flexible plastics/fabrics/paper, either transparent or non-transparent. Different parts can be fused into one piece or be assembled using detachable parts to allow replacement of parts. Different parts/areas can be connected using hinges. The device can be made foldable to reduce space. In some embodiments, the area covering the eyes is open to allow the user to put another part in the open area to cover the empty space, e.g. using detachable transparent glass or plastic pieces, lenses or their own spectacles.

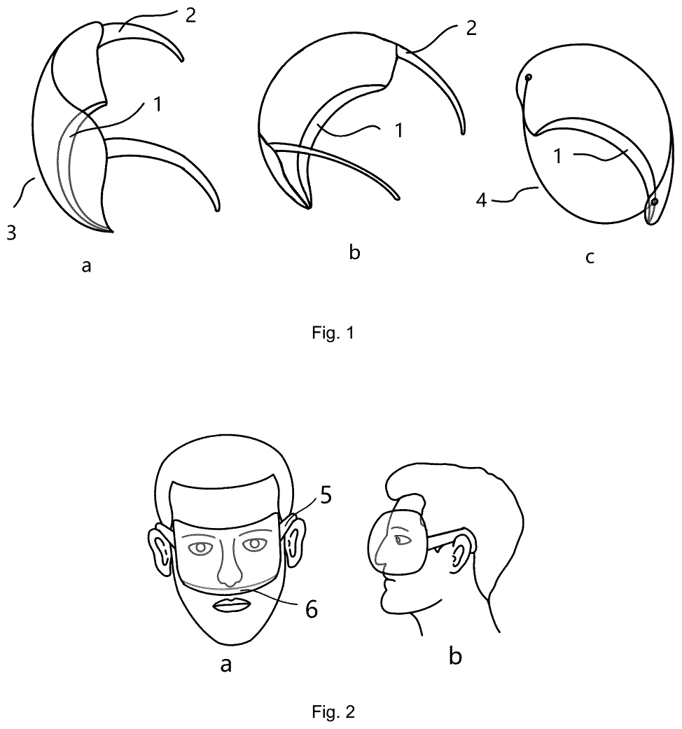

In some embodiments, the device has a convex face covering structure and temples to be mounted on the ears. Examples are shown in FIG. 1. The bottom edge of the face covering 3 folds back to form the breath deflector 1. Temples 2 provide a mounting means to attach to the face. Alternatively, one or more elastic string 4 or band/strap can be used to mount the device to the face as shown in FIG. 1c. The device can be made of transparent material such as polycarbonate. Preferably, the area covering the eyes has an anti-fog coating. The edge of deflector 1 can be wrapped with or coated silicone to provide soft skin contact and/or better air sealing.

FIG. 2 shows the device similar to those in FIG. 1 is worn by the user. Temples 5 and breath deflector 6 are shown in FIG. 2a. The user puts it on his/her head and breathes to deliver the moisture from his/her nose to the eye area to provide a moisturizing effect to the eyes. In a test, the humidity level around the eyes was about 35% before wearing it and reached about 90% after wearing it for around one minute. The dry eye feeling was relieved. When the breath deflector structure in the device is removed, the breath from the nose went downward and the humidity remained below 45% and the patient still felt dry eye symptom.

The side edges of the devices in the current inventions can have a gap between the skin and the face covering, which allows fresh air to enter and be inhaled, providing air circulation. It does not need to seal the air completely between the face covering and face. However, smaller gaps provide quicker and/or better moisture trapping.

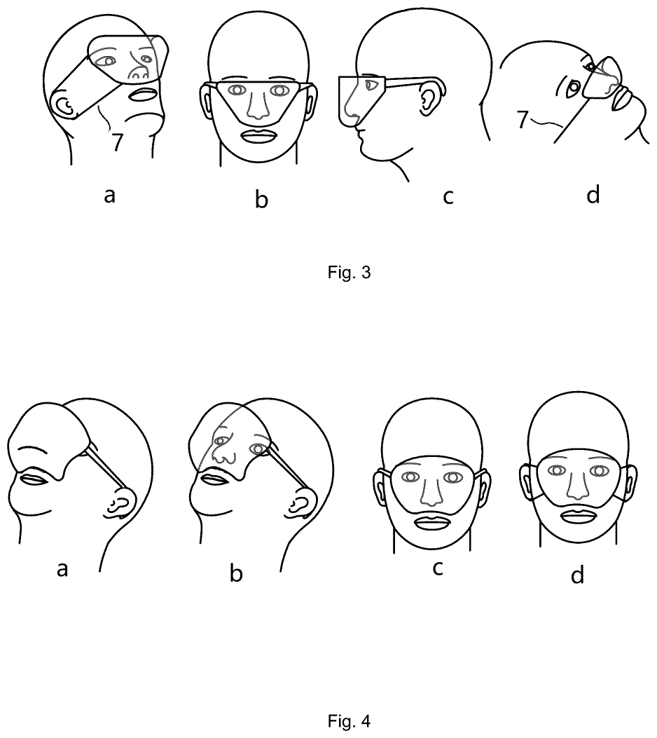

FIG. 3 shows examples of the device made of transparent rigid material such as polycarbonate or polyethylene terephthalate glycol (PETG). Examples in FIGS. 3a and 3d use elastic string 7 or band similar to those used in N95 masks to attach them to the face. Examples in FIGS. 3b and 3c use a structure similar to temples of glasses. Examples in FIGS. 3a, 3b and 3c cover the whole eye area while the device in FIG. 3d only covers area up to the lower eyelid without covering the eyes. A volunteer puts a device in FIG. 3d on his or her head and breathes to deliver the moisture from his or her nose to the eye area to provide a moisturizing effect to the eyes. The humidity level around the eye was 35% before wearing it and reached 85% after wearing it for a minute. The dry eye feeling was relieved.

FIG. 4 shows additional examples of the device with different shapes having either temples or head bands/strings/straps. The example in FIG. 4a is painted to better illustrate the 3D structure and the working device should have the eye area be transparent.

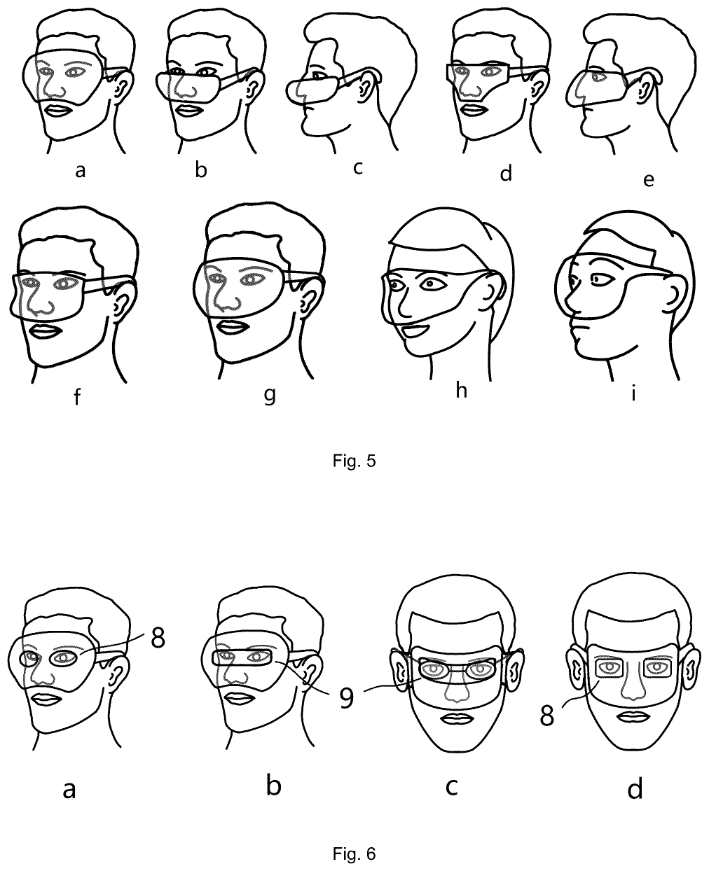

FIG. 5 shows additional examples of the device with different shapes having either temples or head bands/strings/straps worn by the user. The shape of the device and the area covered by the device can vary as long as it can deliver breath moisture to eyes.

FIG. 6 shows additional examples of the device having the area covering eyes open. In FIGS. 6a and 6d, there are two openings 8 in the face coverings directly in front of the eyes having either round/oval (6a) or rectangular shapes (6d). In FIGS. 6b and 6c, there are openings 9 in the face coverings directly in front of the eyes having long rectangular shapes. In FIG. 6c the user can use his own glasses in the open area to cover the eyes while the device is attached to the head with a headband/string. Despite the open areas in the devices, the devices in FIG. 6 can still moisturize the eyes of the volunteer and relieve his dry eye symptoms, which is confirmed by volunteer.

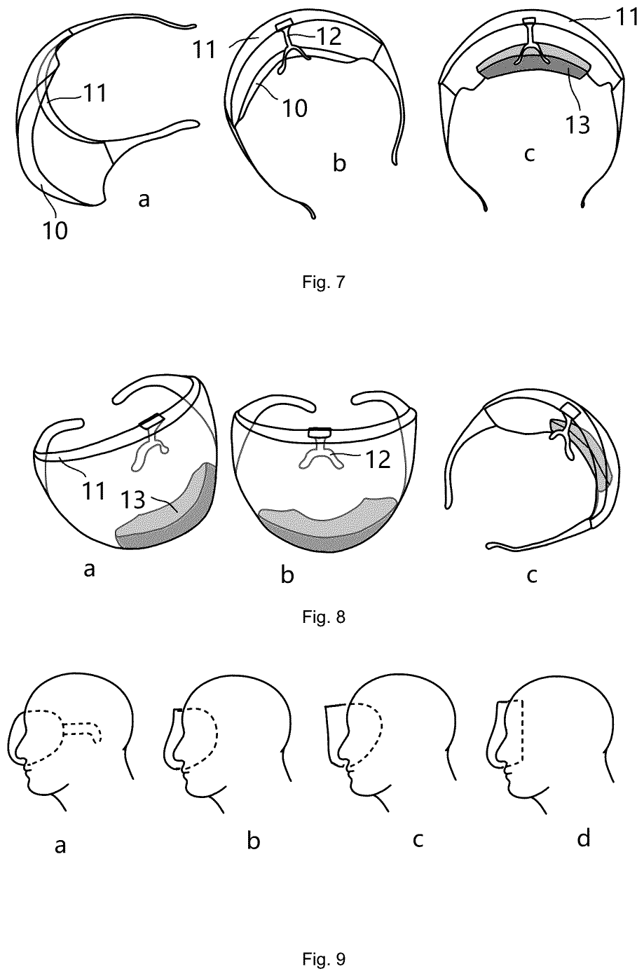

In some embodiments, the device further comprises an airflow blocking means at its upper area that can reduce/prevent the amount of air from exiting from the top. It can be a structure similar to the deflector underneath the nose. FIG. 7 shows examples of the device having both airflow blocking means 11 and breath deflector 10 in the construction. Some of the examples further have a nose clip 12 that can support the device by placing it on the nose. The airflow blocking means, deflector or the edge of them can be wrapped with or coated with or made of a soft material such as rubber, foam or silicone, providing softer contact with the skin and better air sealing. As shown in the FIG. 7c, foam block 13 is used as a breath deflector. FIG. 8 shows different views of a device similar to FIG. 7c. Moisture absorbing material such as foam/fabric block can be placed in the device to function as a moisture reservoir to provide better moisturizing effect.

A volunteer puts the device in FIG. 8 on his head and breathes to deliver the moisture from his nose to the eye area to provide a moisturizing effect to the eyes. The humidity level around the eyes was 35% before wearing it and reached about 100% after wearing it for around 30 seconds. The dry eye feeling was relieved.

FIG. 9 shows longitudinal plane views of examples of the user wearing the device. The dotted line shows the parts of the device not in the longitudinal plane. The breath deflector is a structure that is placed in front of the nose breath air path that can at least partially hinder/deflect/block the exhaled air downward movement and therefore at least partially change the airflow from nose to upward direction. In some embodiments it is preferred that the breath deflector can intercept or block >50% of the area in the exhaled breath path section. The face covering can have a curved structure so its lower part below the nose can serve as breath deflector to block the exhaled breath path.

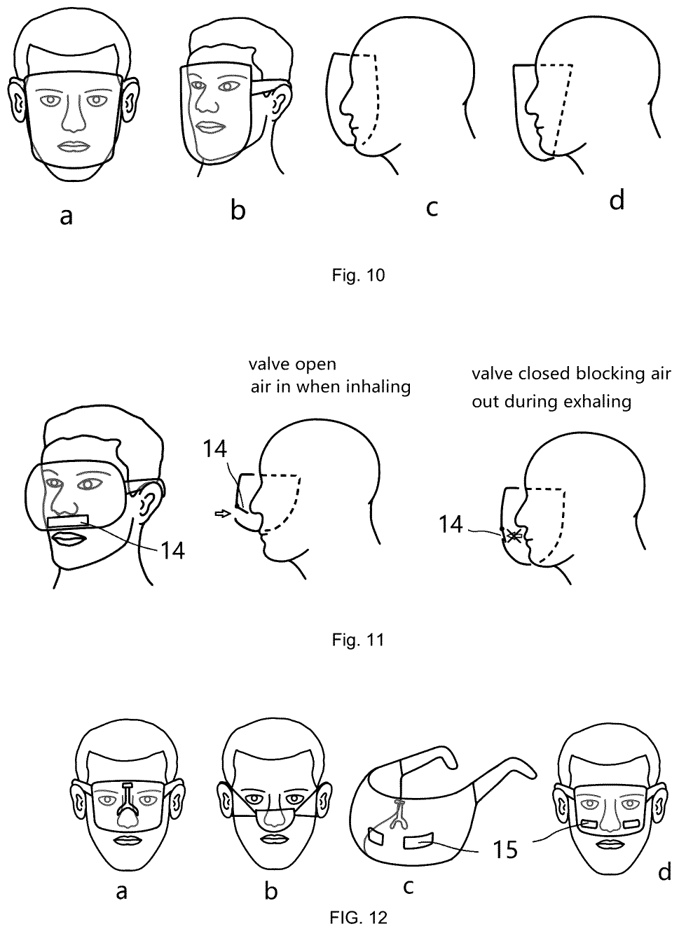

In some embodiments, the face covering of the device can further extend down to or cover the mouth. Examples are shown in FIG. 10 a and b alongside their longitudinal plane views in the FIGS. 10 c and d.

One or more one-way valve/opening can be placed to the area close to the nose to allow fresh air in from the valve/opening to reach the nose during inhalation but not allow the exhaled breath to vent or escape from the valve/opening. Examples are shown in FIG. 11, which has a one way valve 14, though designs or devices other than those depicted can be used as well.

FIG. 12 illustrates additional examples of the device being used by the user. The device can have additional vents in the face cover to allow the exhaled breath to exit to reduce or control the moisture level inside. The vent can be one or more adjustable opening such as a window or sliding door type structure. The user can adjust the openness to adjust the moisture level inside. Examples are shown in FIGS. 12c and d, which have two windows 15 (vent) along the nose to adjust moisture level. The windows can have a sliding door structure/mechanism with sliding rail, so the user can adjust the area of openness of the window to control the moisture level inside. The more the vent area opened, the lower the moisture inside.

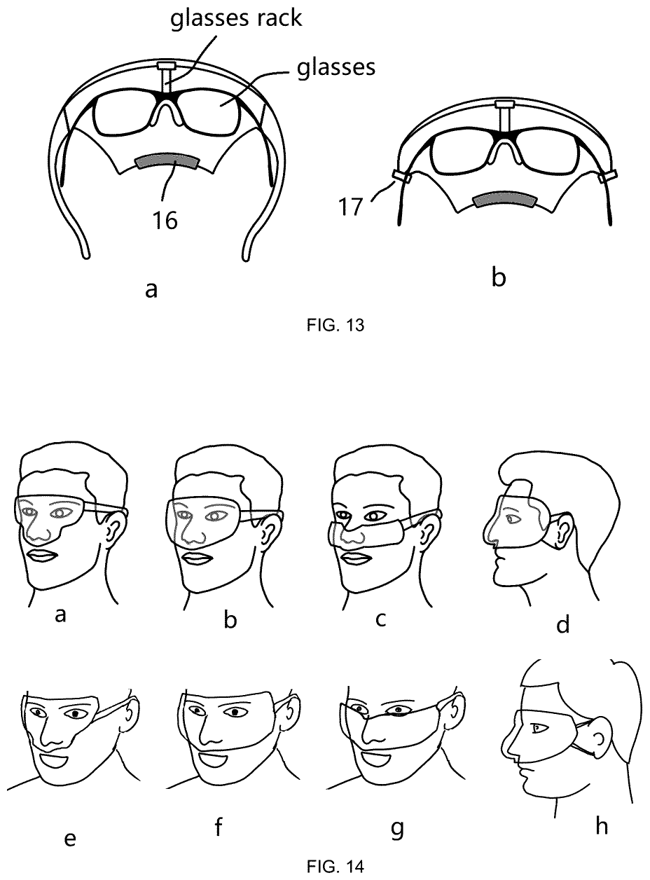

In some embodiments, a glasses rack can be incorporated into the device to allow the user put their glasses inside the device. FIG. 13 illustrates examples of the device having a glasses rack and foam 16 as deflector. The device in FIG. 13a has its own temples to be mounted on the ear of the user. In some embodiments, the device can use the temples of the glasses to mount it on the ear of the user. The device can have a lock structure (e.g. a tie, a rubber band, a groove) that can attach it to the temples of the glasses. FIG. 13b illustrates an example of the device having a locking or mounting structure 17 that is mounted to the temples of the glasses.

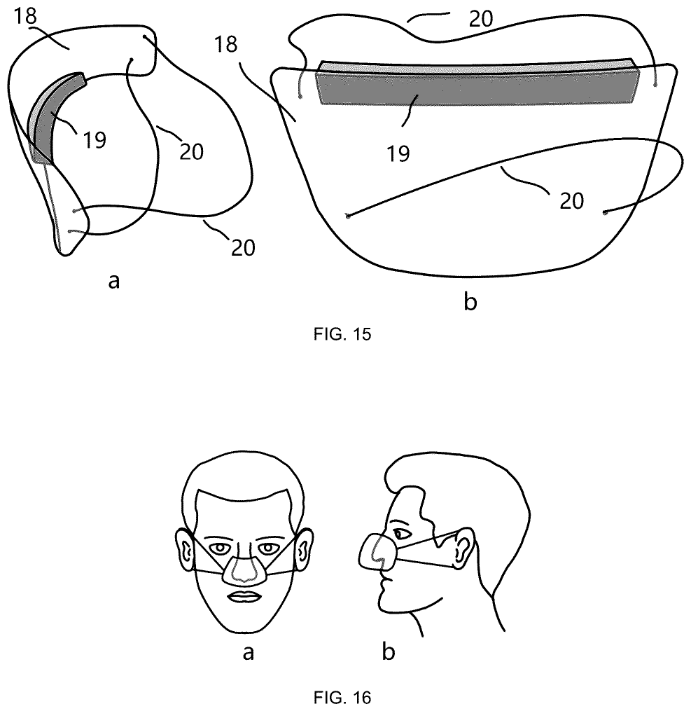

In some embodiments, the area in front of the nose can protrude from the rest of the face covering structure. The face covering can include a nose protrusion area or conic structure in front of the nose to surround or hold the nose. Examples are shown in FIG. 14.

In some embodiments, the device uses flexible/bendable material to form the face covering. FIG. 15 shows an example of the device using a flexible PET sheet 18 as face cover. It has breath deflector foam 19 and head belts/strings 20. When it is attached to the head using the elastic head belt, it folds to a curved shape as shown in the left figure to fit the shape or contour of the face. When it is placed on a surface and allowed to relax with no restraining belt, it is flat.

Alternatively, in some embodiments the center part of the face covering can be made of rigid material and the surround/side parts can be made of flexible or soft material to provide better skin contact.

In some embodiments the device or the area facing the eyes is made of transparent material with blue light and/or UV blocking property to further protect eyes.

In some embodiments the device of the current invention has both elastic string/band type of mounting means and mounting means with structure similar to temples of glasses to mount it to the face, the elastic string/band and temples of glasses type structure can be detachable so the user can chose freely. It can has one or two or more elastic string or band type of mounting means to secure its position on the face.

Similar devices can also be used to relieve dry nose symptoms, and to hydrate the nose and facial skin. Therefore the current invention also discloses method and device to relieve dry nose symptoms, and to hydrate respiratory ducts and facial skin. The device to relieve dry nose symptoms and hydrate respiratory duct can be the same or similar to the device to hydrate eyes. In some embodiments, it is similar to the device to hydrate eyes except it only covers the nose area. Examples are shown in FIG. 16. It has a nose cone structure that can be placed on the nose, which can also have moisture absorbing material on the inner surface such as content, fabrics to retain moisture from the breath. User can put it on his nose and breathe to hydrate the nose as shown in the figure.

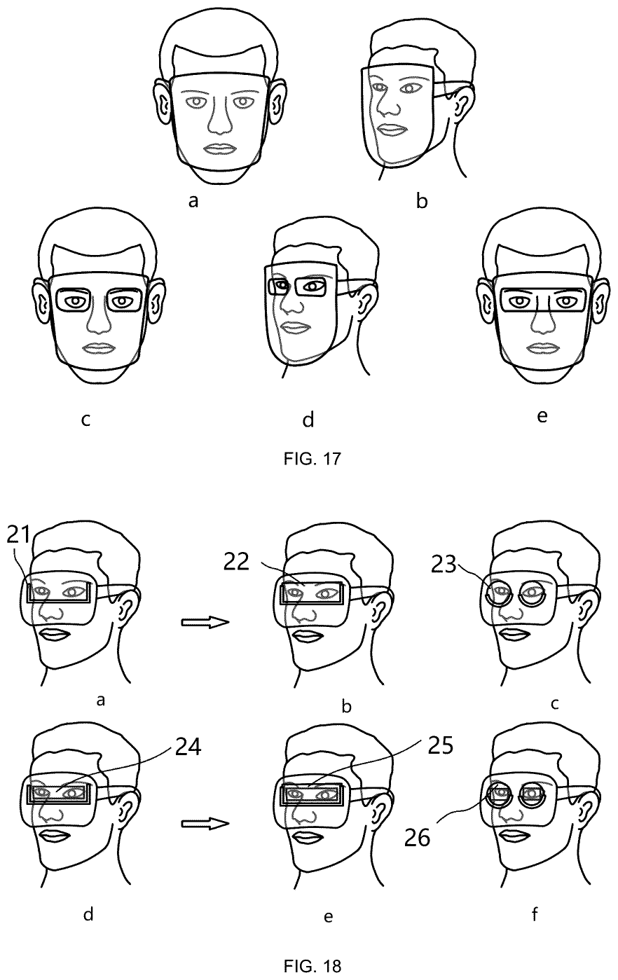

The device to hydrate facial skin can be the same or similar to the device to hydrate eyes. In some embodiments, it is similar to the device to hydrate the eyes except it covers the full face. Examples are shown in FIG. 17. In some embodiments, the area in front of the eyes is open so it will not affect vision, as shown in examples in FIG. 17 c, d and e.

In some embodiments, the face cover of the devices of the current invention can have a meaning to allow external glass/lens to be incorporated. As shown in FIG. 18, the see through area directly in front of the eyes has a groove/pocket frame 21 or other snap on means that allow a piece of anti-scratch glass plate 22 to be inserted. The glass plate can be mass produced the same to fit different shape of face covering as long as shape/size of the frame 21 is unchanged. Lens 23 can be inserted instead as shown in FIG. 18c. The area 24 directly in front of the eyes can be open as shown in the FIG. 18 d-e. Glass plate 25 or lens 26 can be inserted in the groove/pocket frame in front of the opening 24. Two lenses (e.g. those in glasses having index) instead of one plate can be inserted as shown in FIGS. 18c and 18f.

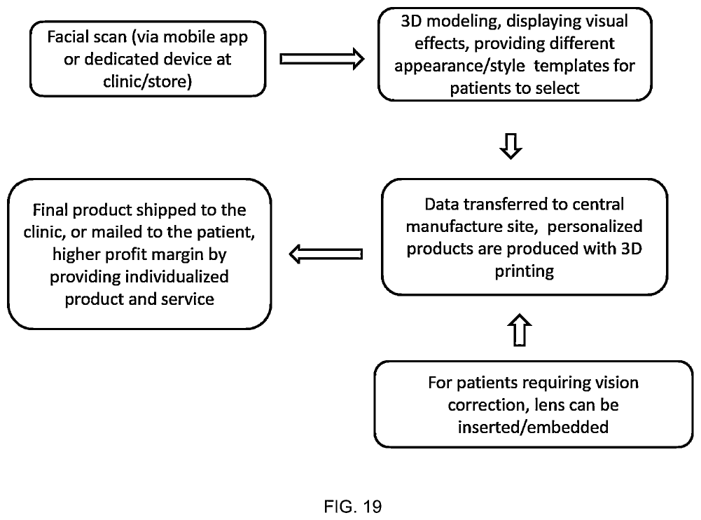

In some embodiments, the face cover of the devices of the current invention can also be custom made or tailored to the user who needs the device, so as to better fit with the shape and contour of the user's face, providing a greater fit. This can also allow the device to be better suited to the user's preferences, by giving the user a greater level of choice and customizability for the preferred shape, style, or design of the device. An operator or the user him or herself can use a scanning application to map out the shape and contour of the user's facial structure. The information can be then sent to a production center to be used to create a custom fit device for that user. The mode of creation can involve 3D printing, or other means of manufacture as well. Certain parts may be individually created or manufactured. Alternatively, certain structures, such as the see through area directly in front of the eyes (e.g. a piece of glass with anti-fog coating), may be manufactured the same with mass production means and then customized by attaching other parts, or be embedded into a customized face covering structure. In some embodiments, a user can use a cell phone app to scan his face or go to a reading center (e.g. a local store or physician's office) to scan his face (e.g. using a scanner such as 3D laser scanner) to obtain the information of face shape and contour. Furthermore the information can be used to create a 3D model. A software or cellphone APP can attach different type/shape/style of devices to the 3D model and present the visual result to the user to allow the user to select the type/shape/style he prefers. The facial information with optional type/shape/style selection is sent to a production center to manufacture a device to fit the user's facial structure and selection. The final product will be sent back to the user. Example of the procedure is shown in FIG. 19. It shows the method to make custom fit device for a user.

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known mechanics, instruments, methods and skills. A skilled person can easily find the knowledge from textbooks such as the textbooks, scientific journal papers and other well-known reference sources.

1. A device for relieving dry eye symptoms in a subject in need, said device comprising a face covering over the nose, a breath deflector and a means that can attach the device to the face.

2. The device of claim 1, wherein the face covering covers the nose and not covers the mouth.

3. The device of claim 1, wherein the breath deflector is underneath the nose.

4. The device of claim 1, wherein the means that can attach the device to the face is selected from head band, string, strap, belt, temple and their combinations.

5. The device of claim 1, wherein the face covering has anti-fog coating.

6. A method for relieving dry eye symptoms in a subject in need thereof, the method comprising delivering the subject's exhaled breath to his or her eye area to provide moisture there to relieve dry eyes by attaching a device to the face, wherein the device comprises a face covering over the nose, a breath deflector and a means that can attach the device to the face.

7. The method of claim 6, wherein the face covering covers the nose and not covers the mouth.

8. The method of claim 6, wherein the breath deflector is underneath the nose.

9. The method of claim 6, wherein the face covering has anti-fog coating.
